(19) United States
(12) Patent Application Publication
(10) Pub. No.: US 2016/0083326 A1
(43) Pub. Date: Mar. 24, 2016

(54) Cost Effective Biorefining Process in Which the Product Separation Is Very Easy

(71) Applicants: Zuolin Zhu, San Diego, CA (US); Jonathan Zhu, San Diego, CA (US)

(72) Inventors: Zuolin Zhu, San Diego, CA (US); Jonathan Zhu, San Diego, CA (US)

(21) Appl. No.: 14/492,319

(22) Filed: Sep. 22, 2014

(51) Int. Cl. C07C 51/42; C07C 1/22; C10L 1/04; C07C 4/22; C07C 2/66; C07C 51/00

(52) U.S. Cl. CPC C07C 51/42; C07C 2/66; C07C 51/00; C10L 1/04; C07C 4/22; C07C 1/22

(57) ABSTRACT

A process for converting lignocelluloses selectively into liquid fuels and fine chemicals uses one step of reaction and one step of isolation and separation. The process has the fewest steps of operation for converting biomass into products compared with all known biorefining processes.

### FIELD OF THE INVENTION

The present invention is related to a novel process of product isolation and separation for the quantitative catalytic depolymerization of lignocelluloses in a one-pot reaction. More specifically, the present invention is related to a process for product separation using only a one-step filtration operation.

### DESCRIPTION OF RELATED ART

Although there have been many processes invented up to now, none of them are cost effective. Many known methods for the biorefining of lignocelluloses have been proved commercially impractical due to high costs of operation. Thermal-chemical methods, such as liquefaction, pyrolysis, etc., have shown no product selectivity. The produced mixture contains phenols, aldehydes, alcohols, acids, alkanes, olefins, esters, ethers, etc. All known methods have continued to show the formation of black tar and low yields of liquid products. Bio-chemical methods require component separation/isolation and purification, which results in a huge loss of organic carbon in percentage and a slow rate of cellulose depolymerization. All known methods that involve lignin isolation first, followed by depolymerization and deoxygenation, have been proved to be commercially ineffective (Chem. Rev., 2010, volume 110, 3552-3599). USDOE Report to White House (PNNL-16983, October, 2007) predicts that a commercially practical process requires a one-step reaction for converting lignin to small molecular aromatics or transportation liquid fuels, without a significant loss of organic carbons. The report emphasized that such a process requires long-term R&D efforts—“Long-term” meaning beyond decades, requiring significant, new, fundamental knowledge and technological developments.

Our invented catalytic method of one-pot hydrolytic depolymerization of lignocelluloses meets the qualification of the commercial process predicted by the USDOE report—There is no need for lignin separation. Lignin is co-depolymerized into small molecular aromatics with other components of lignocelluloses such as cellulose, hemicellulose, proteins, etc. Unlike lignin itself, these small molecular aromatics can be converted into liquid transportation fuels using a one-step hydrocracking or alkylation reaction.

Easy operation methods for product isolation are critical for a cost-effective process. In practice, processes with fewer operation steps are more cost-efficient. The method of catalytic one-pot hydrolytic depolymerization of lignocelluloses converts all organic polymers of lignocelluloses into two kinds of products: small molecular aromatics, and small organic acids. There are several known methods in the industry for isolating small molecular aromatics and small organic acids from aqueous solution, such as absorption, distillation, extraction, etc. These methods, however, have drawbacks. For example, the most popular isolation method for small molecular aromatics from aqueous solution is absorption with resins. This method includes two steps: absorption and de-absorption. There are many problems with this process, such as the constant clogging of columns, prevention of de-absorption reaching 100%, unavoidable resin defouling, etc. Generally, de-absorption consists of washing with another solvent, followed by removing the solvent using distillation, thus adding more cost to the process due to excess work. Another method for isolating small molecular aromatics from aqueous solution is distillation. Because all small molecular aromatics have higher boiling points than water, distillation requires all water to be distilled first; however, the distillation of water is expensive. In addition, some of the small molecular aromatics are azeotropic with water leading to the loss of small molecular aromatics. Extraction is another method for isolating small molecular aromatics from aqueous solution. Although the extraction percentage is mostly very high, there are two drawbacks to this method. First, some extracting solvent will be lost due to its solubility in water; second, the extracting solvent has to be recovered through a costly process. There are also several conventional methods available for isolating small organic acids from aqueous solution, such as distillation, extraction, esterification and distillation (reacting distillation), SMB (simulated moving bed) chromatographic method, absorption with ionic resins, etc. These methods all have drawbacks. The disadvantages of distillation, extraction, and absorption are the same as those for isolating small molecular aromatics. Reacting distillation produces an ester as the product, requires a high cost of operation, and creates excess expenses in order to convert the ester back to acid. SMB is always too expensive to use for commodity chemical products. A novel easy-operating separation process is crucial for the profitability of our invented catalytic method of one-pot hydrolytic depolymerization of lignocelluloses.

### SUMMARY OF THE INVENTION

It is a primary object of the present invention to provide a novel biorefining process, in which, the isolation and separation of two kinds of products requires only a one step operation. The two kinds of products are small molecular aromatics and small organic acids.

It is another object of the present invention to provide a process for producing transportation liquid fuels from lignin in a one-step reaction. In comparison with that proposed by USDOE report (PNNL-16983, October, 2007). This USDOE report does not include lignin isolation and purification. One-step reaction means that the transformation from pure lignin to desired products (transportation liquid fuels, or alkylated benzenes) is done in one step.

It is a key object of the present invention to provide a process for producing transportation liquid fuels and fine chemicals that is reliable and cost-effective. Cost-effective means that the manufacturing costs of the products from lignocelluloses are comparable or even lower with that of petro-chemical processes.

To achieve the foregoing objects, and in accordance with the invention as embodied and described herein, a novel isolation process is provided for isolating small molecular aromatics and small organic acids from aqueous solution in a one step operation. Three years after trying out all conventional isolation methods through countless experiments, the inventors discovered that when oxides or hydroxides of alkaline earth metals are used in the catalytic one-pot hydrolytic depolymerization reaction, the product of small molecular aromatics is solid and can be isolated from aqueous solution by a one-step filtration with the solution temperature maintained above fifty degrees Celsius. Then, the product of small organic acids becomes solid when the solution temperature is cooled down to below ten degrees Celsius. Therefore, the product of small organic acids can also be isolated from aqueous solution by a one-step filtration. In addition to the oxides or hydroxides of alkaline earth metals, the oxides or hydroxides of transition metals are also discovered to be able to fulfill the one-step isolation and separation.

The process of the invention has the advantages of being cost-effective and easy to operation, unlike conventional methods, such as distillation, extraction, esterification and distillation (reacting distillation), SMB (simulated moving bed) chromatographic method, absorption with ionic resins, etc.

### BRIEF DESCRIPTION OF FIGURES

FIG. 1 illustrates the process scheme of the invented process.

FIG. 2 illustrates the isolated product of small organic acid via a one-step filtration operation.

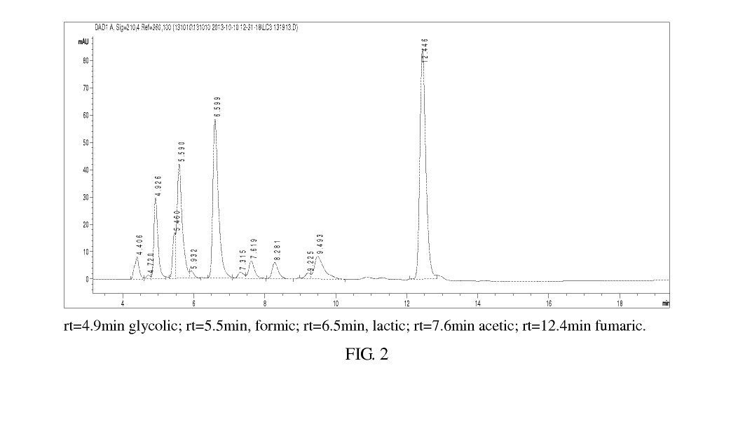

FIG. 3 illustrates the LCMS of the isolated product of small molecular aromatics via one-step filtration operation.

FIG. 4 illustrates the LCMS of the isolated product of small molecular aromatics via extraction followed by removing solvent under reduced pressure. Molecular weight is increased compared with the product using pressed filtration method.

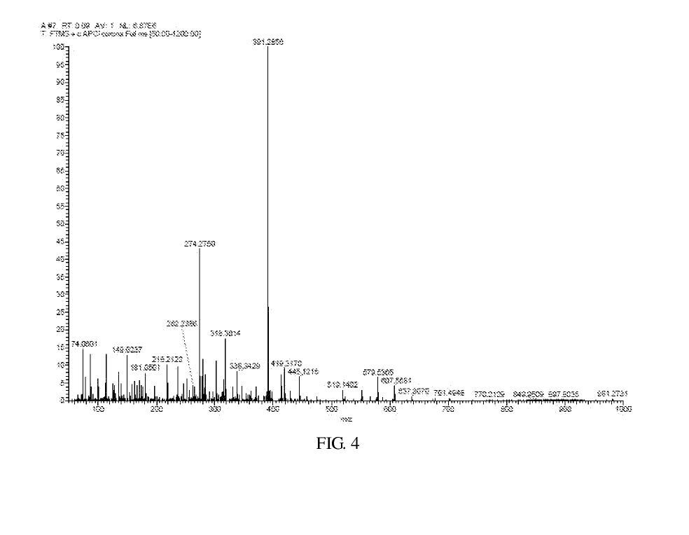

### DETAILED DESCRIPTION OF THE INVENTION

The present invention provides a novel cost-effective process for the conversion of renewable lignocelluloses to small organic molecules in an almost quantitative yield. The invented process has the fewest number of operation steps compared with all known processes. There is only a one reaction step for depolymerizing all organic components of lignocelluloses into small organic molecules with high product selectivity. The “high product selectivity” means that there are only two kinds of products obtained from the catalytic one-pot hydrolytic depolymerization of lignocelluloses: small molecular aromatics and small organic acids. The isolation and separation of two kinds of products is also completed in a one step operation. The process of the invention provides the basis for cost-effective manufacturing technology aimed at the production of transportation liquid fuels and fine chemicals from lignocelluloses with an almost quantitative yield.

The present invention is directed to a two-step process for converting all kinds of lignocelluloses into small organic molecules, a novel process with a potential to be the most economic biorefining technology.

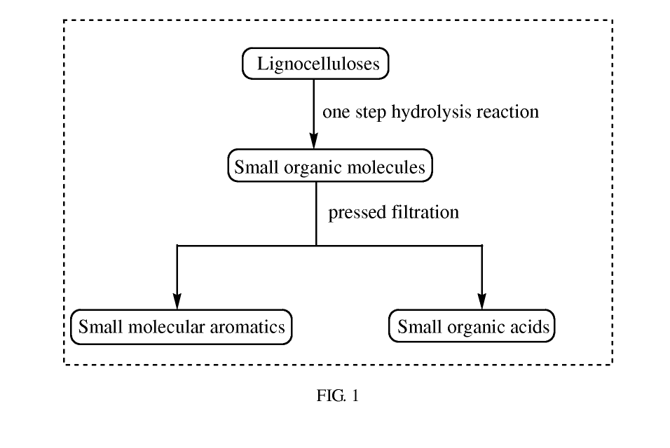

In the first step of the process of the invention, as described in FIG. 1, lignocelluloses is subjected to a one-step hydrolytic depolymerization in a one-pot reaction. All organic components of lignocelluloses, such as cellulose, lignin, hemicellulose, protein, etc., are depolymerized selectively into two kinds of small organic molecules. The reaction is fast with good product selectivity: neither gasification nor black tar formation is observed during the catalytic one-pot hydrolytic depolymerization reaction.

Oxides or hydroxides of metals are added into the hydrolysis reaction to facilitate the one step isolation and separation. It is almost impossible to fulfill one step isolation and separation without the oxides or hydroxides of metals.

The metals in oxides or hydroxides are alkaline earth metals and transition metals. The simple rule for the selection of the metals is the availability and price of the oxides or hydroxides. For example, when the biorefining facility is near a steel manufacturing plant, iron(II) oxide is preferred, because slag is easy to obtain and the price is very reasonable. When the biorefining facility is near a lime production plant or located in an area where limestone resource is very rich, calcium oxide or calcium hydroxide is preferred.

The ratio of biomass to oxide or hydroxide depends on the nature of lignocelluloses. In principal, the higher the content of cellulose and hemicellulose, the higher the amount of oxide or hydroxide needed. For example, reed contains 49% cellulose, 30% hemicellulose, and 10% lignin. Catalytic one-pot hydrolytic depolymerization of 50 grams of reed (dried) requires a one molar equivalent of hydroxide anion. Pine needle contains 43% lignin, 53% cellulose and hemicellulose. A one molar equivalent of hydroxide anion can fulfill the completed catalytic one-pot hydrolytic depolymerization of 100 grams of pine needles. The requirement of metal oxide or hydroxide for the completion of biomass hydrolytic depolymerization, expressed as the one molar equivalent of hydroxide anion, is in the range of 50 grams of biomass/one molar equivalent of hydroxide anion to 100 grams of biomass/one molar equivalent of hydroxide anion.

As used herein, the term “lignocelluloses” and “biomass” are interchangeable, it refers to biomass products produced by plants (herbaceous plant, and ligneous plant), such as leaves, stalks, roots, skins, etc. The lignocelluloses refers to materials that has the major components of lignin, and/or cellulose, and/or hemicellulose.

In the second step of the process of the invention, as described in FIG. 1, products are isolated and separated by filtration. In the present invention, there are only two kinds of products obtained from the catalytic one-pot hydrolytic depolymerization of lignocelluloses: small molecular aromatics and small organic acids. With the help of metal oxides or hydroxides, when the temperature is higher than fifty degrees Celsius, small molecular aromatics are solid in aqueous solution, while, small organic acids are soluble in aqueous solution.

In the present invention, the isolation and separation of small molecular aromatics is carried out with the temperature of product solution maintained above fifty degrees Celsius. Pressed filtration is preferred, so the water content of the solid can be lower than 40%, or even lower than 30%.

According to the invention, after the isolation and separation of small molecular aromatics, the filtrate can be used as the solvent for the catalytic one-pot hydrolytic depolymerization in order to increase the percentage content of small organic acids in aqueous solution.

According to the invention, the isolation and separation of small organic acids is carried out with the solution temperature controlled below ten degrees Celsius. After the isolation and separation of small molecular aromatics, the temperature of product solution is cooled to below ten degrees Celsius. The pressed filtration is preferred, so the water content of the solid products can be lower than 40%, or even lower than 30%.

According to the invention, the isolated product of small molecular aromatics can be converted into high quality transportation liquid fuels using a one step reaction. Alkylation and hydrocracking are preferred methods for the transformation. Alkylation can be fulfilled with small alcohols or with dimethyl sulfate or dimethyl carbonate. Hydrocracking is similar to that of petrochemical method with Co—Mo or Ni—Mo catalytic systems.

According to the invention, the isolated product of small organic acids are naturally degradable and can be used as green deicer.

The following examples are included to demonstrate the preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Analysis of organic acids: The organic acids are analyzed using the following methods and conditions: HPLC, Agilent 1200, column: SB-AQ, 5 μm, 4.60×250 mm; mobile phase: 0.025M, pH=2.5 phosphate buffer solution, flow rate: 0.6 ml/min for 0-10 min, 0.6 ml/min to 1.2 ml/min from 10-15 min, 1.2 ml/min to 0.6 ml/min from 15-30 min, then at 0.6 ml/min for 45 minutes. A DAD detector is used at wavelength 210 nm and a column temperature is kept at 30° C. Sample size is 5 μl and all organic acids have a retention time of less than 14 minutes.

All volatile products such as phenols are analyzed using GCMS: Agilent 7890 GC with Agilent 5975C MS; Column: DB-5, 30 m×0.25 mm×0.25 μm; Injector: 10:1 split, 250° C.; Carrier gas: helium at 1.0 ml/min; Temperature: 60° C. initial, hold 5 min, ramp at 2° C./min to 200° C., hold 5 min, ramp at 2° C./min to 280° C., hold 5 min; Detector: Agilent 5975C; transfer line temperature 280° C.; ion source temperature 230° C.; quadruple temperature 150° C.; mass range 40-500 ug; ionization voltage 70 ev; Injection volume 0.2 μl.

Molecular mass of small molecular aromatics: Source Type: APCI

| | |
|---|---|
| APCI Vaporizer Temp (° C.): 500.00 | Sheath Gas Flow (arb): 50.00 |
| Aux Gas Flow (arb): 10.00 | Sweep Gas Flow (arb): 5.00 |
| Capillary Temp (° C.): 350.00 | I Spray voltage(kv): 3.5 |
| Capillary voltage (v): −31 | Tube lens (V): −90 |

### EXAMPLE 1

To a 1 liter stainless steel autoclave equipped with mechanical stirrer, add 750 ml of distilled water, 112 g of chopped dry grape tree branches (2-10 mm size, containing 38% cellulose, 20% hemicellulose, 29% lignin, and 13% others), 42 g of calcium oxide, and 1 g of disulfonate sodium salt of 9,10-dihydroxyanthracene. The autoclave is sealed, purged with nitrogen three times, then filled with nitrogen at a pressure of 0.2-1 MPa, heated to 180° C. and maintain at this temperature for ten minutes. The temperature is then increased to 250° C. and stirred at this temperature for 50 min. After cooling to a temperature below 90° C., a brown color liquid mixture is obtained (the color slowly turns deeper after the mixture is exposed to air).

The brown color liquid mixture is then cooled down to a temperature below 10 degrees Celsius with stirring. Pressed filtration is carried out with the temperature controlled below 10 degrees Celsius. Brown color solid obtained is 188.8 gram with 30% of water content.

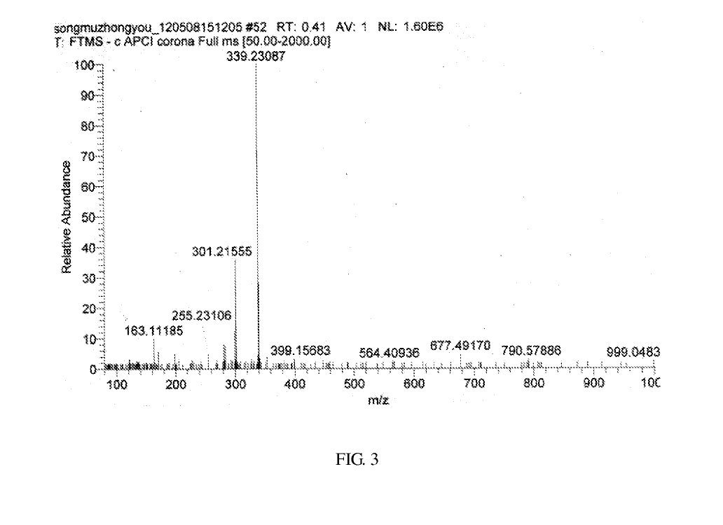

The brown color solid is suspended in a mixture of 200 ml of MTBE and 100 ml of water, 95% of sulfuric acid is used to adjust pH=4.3 with vigorous stirring. Pressed filtration is carried out to remove all solids. The isolated organic phase is concentrated to obtain 35.6 g of deep brown color sticky product. The moisture content of this product is less than 1%. The yield of small aromatics obtained is 101% based on lignin weight. Molecular mass analysis shows it is a mixture (FIG. 3), most of them having molecular weights between 162 to 340. Elemental analysis shows the oxygen content of this product is 18%, far below the oxygen content of lignin (˜36%).

The aqueous phase is then acidified again, 95% of sulfuric acid is used to adjust pH=1.0 with vigorous stirring. Pressed filtration is carried out to remove all solids. HPLC analysis of the product shows that only several small organic acids are formed, such as formic acid, glycolic acid, acetic acid, lactic acid, succinic acid, etc. It contains 40 grams of lactic acid, 13 grams of glycolic acid, 14.1 grams of formic acid, 1.5 grams of acetic acid, 3.2 grams of succinic acid, and the rest 2-4 grams of the products are 2-hydroxyl isobutyric acid, fumaric acid, etc. The yield is about 102% based on the weight of cellulose and hemicellulose.

### EXAMPLE 2

To a 1 liter stainless steel autoclave equipped with mechanical stirrer, add 750 ml of distilled water, 112 g of chopped dry grape tree branches (2-10 mm size, containing 38% cellulose, 20% hemicellulose, 29% lignin, and 13% others), 42 g of calcium oxide, and 1 g of disulfonate sodium salt of 9,10-dihydroxyanthracene. The autoclave is sealed, purged with nitrogen three times, then filled with nitrogen at a pressure of 0.2-1 MPa, heated to 180° C. and maintain at this temperature for ten minutes. The temperature is then increased to 250° C. and stirred at this temperature for 50 min. After cooling to a temperature below 90° C., a brown color liquid mixture is obtained (the color slowly turns deeper after the mixture is exposed to air). The temperature is maintained between 50-70 degrees Celsius, and pressed filtration is carried out to obtain 58 g of brown color solid A. Analysis results show the brown color solid A contains 28.3% water.

The filtrate is then cooled down to a temperature below 10 degrees Celsius with stirring. Pressed filtration is carried out with the temperature controlled below 10 degrees Celsius. Brown color solid B obtained is 132 gram with 31% of water content.

The brown color solid A is suspended in 200 ml of anhydrous methanol, 95% of sulfuric acid is used to adjust pH=3.0 with vigorous stirring. Pressed filtration is carried out to remove all solids. The filtrate is concentrated to obtain 35 g of deep brown color sticky product. The moisture content of this product is less than 1%. The yield of small aromatics obtained is 101% based on lignin weight. Molecular mass analysis shows it is a mixture (same as shown by FIG. 3), most of them having molecular weights between 162 to 340. Elemental analysis shows the oxygen content of this product is 18%, far below the oxygen content of lignin (˜36%).

The brown color solid B is suspended in 300 ml of anhydrous acetone, 95% of sulfuric acid is used to adjust pH=1.0 with vigorous stirring. Pressed filtration is carried out to remove all solids. The filtrate is concentrated to obtain 75 g of light brown color liquid product. The moisture content of this product is 2.3%. The yield of small organic acids is 103% based on the weight of cellulose and hemicellulose. HPLC analysis of the product shows that only several small organic acids are formed, such as formic acid, glycolic acid, acetic acid, lactic acid, succinic acid, etc. It contains 40 grams of lactic acid, 13 grams of glycolic acid, 14.1 grams of formic acid, 1.5 grams of acetic acid, 3.2 grams of succinic acid, and the rest are 2-hydroxyl isobutyric acid, fumaric acid, etc. Experiments are carried out followed the exact procedure with equal molar amount of calcium hydroxide to calcium oxide, same results are obtained.

### EXAMPLE 3

To a 1 liter stainless steel autoclave equipped with mechanical stirrer, add 750 ml of distilled water, 112 g of chopped dry reed (2-10 mm size, containing 49% cellulose, 30% hemicellulose, 10% lignin, and 11% others), 63 g of calcium oxide, and 1 g of disulfonate sodium salt of 9,10-dihydroxyanthracene. The autoclave is sealed, purged with nitrogen three times, then filled with nitrogen at a pressure of 0.2-1 MPa, heated to 180° C. and maintain at this temperature for ten minutes. The temperature is then increased to 250° C. and stirred at this temperature for 50 min. After cooling to a temperature below 90° C., a brown color liquid mixture is obtained (the color turns deeper slowly after the mixture is exposed to air). The temperature is maintained between 50-70 degrees Celsius, and pressed filtration is carried out to obtain 18 g of brown color solid A. Analysis results show the brown color solid A contains 22% water.

The filtrate is then cooled down to a temperature below 10 degrees Celsius with stirring. Pressed filtration is carried out with the temperature below 10 degrees Celsius. Brown color solid B obtained is 186 gram with 33% of water content.

The brown color solid A is suspended in 60 ml of anhydrous methanol, 95% of sulfuric acid is used to adjust pH=3.0 with vigorous stirring. Pressed filtration is carried out to remove all solids. The filtrate is concentrated to obtain 13 g of deep brown color sticky product. The moisture content of this product is less than 1%. The yield of small aromatics obtained is 100% based on lignin weight. Molecular mass analysis shows it is a mixture similar to that obtained from the catalytic one-pot hydrolytic depolymerization of grape tree branches, most of them having molecular weights between 162 to 340. Elemental analysis shows the oxygen content of this product is 15%, far below the oxygen content of lignin (˜36%).

The brown color solid B is suspended in 350 ml of anhydrous acetone, 95% of sulfuric acid is used to adjust pH=1.0 with vigorous stirring. Pressed filtration is carried out to remove all solids. The filtrate is concentrated to obtain 99 g of light brown color liquid product. The moisture content of this product is 3.1%. The yield of small organic acids is 101% based on the weight of cellulose and hemicellulose. HPLC analysis of the product shows that only several small organic acids are formed, such as formic acid, glycolic acid, acetic acid, lactic acid, succinic acid, etc. It contains 54 grams of lactic acid, 17.4 grams of glycolic acid, 17.88 grams of formic acid, 2.1 grams of acetic acid, 4.3 grams of succinic acid, and the rest are 2-hydroxyl isobutyric acid, fumaric acid, etc.

### EXAMPLE 4

To a 1 liter stainless steel autoclave equipped with mechanical stirrer, add 750 ml of distilled water, 112 g of chopped dry pine needles (2-10 mm size, containing 53% cellulose and hemicellulose, 43% lignin, and 4% others), 32 g of calcium oxide, and 1 g of disulfonate sodium salt of 9,10-dihydroxyanthracene. The autoclave is sealed, purged with nitrogen three times, then filled with nitrogen at a pressure of 0.2-1 MPa, heated to 180° C. and maintain at this temperature for ten minutes. The temperature is then increased to 250° C. and stirred at this temperature for 50 min. After cooling to a temperature below 90° C., a brown color liquid mixture is obtained (the color slowly turns deeper after the mixture is exposed to air). The temperature is maintained between 50-70 degrees Celsius, and pressed filtration is carried out to obtain 73 g of brown color solid A. Analysis results show the brown color solid A contains 26% water.

The filtrate is then cooled down to a temperature below 10 degrees Celsius with stirring. Pressed filtration is carried out with the temperature below 10 degrees Celsius. Brown color solid B obtained is 103 gram with 28% of water content.

The brown color solid A is suspended in 200 ml of anhydrous methanol, 95% of sulfuric acid is used to adjust pH=3.0 with vigorous stirring. Pressed filtration is carried out to remove all solids. The filtrate is concentrated to obtain 51 g of deep brown color sticky product. The moisture content of this product is less than 1%. The yield of small aromatics obtained is 101% based on lignin weight. Molecular mass analysis shows it is a mixture similar to that obtained from the hydrolysis of grape tree branches, most of them having molecular weights between 162 to 340. Elemental analysis shows the oxygen content of this product is 19.3%, far below the oxygen content of lignin (˜36%).

The brown color solid B is suspended in 300 ml of anhydrous acetone, 95% of sulfuric acid is used to adjust pH=1.0 with vigorous stirring. Pressed filtration is carried out to remove all solids. The filtrate is concentrated to obtain 68 g of light brown color liquid product. The moisture content of this product is 2.6%. The yield of small organic acids is 101% based on the weight of cellulose and hemicellulose. HPLC analysis of the product shows that only several small organic acids are formed, such as formic acid, glycolic acid, acetic acid, lactic acid, succinic acid, etc. It contains 35 grams of lactic acid, 11 grams of glycolic acid, 12 grams of formic acid, 1.1 grams of acetic acid, 1.9 grams of succinic acid, and the rest are 2-hydroxyl isobutyric acid, fumaric acid, etc.

### EXAMPLE 5

To a 1 liter stainless steel autoclave equipped with mechanical stirrer, add 750 ml of distilled water, 112 g of chopped dry grape tree branches (2-10 mm size, containing 38% cellulose, 20% hemicellulose, 29% lignin, and 13% others), 120 g of iron(II) oxide, and 1 g of disulfonate sodium salt of 9,10-dihydroxyanthracene. The autoclave is sealed, purged with nitrogen three times, then filled with nitrogen at a pressure of 0.2-1 MPa, heated to 180° C. and maintain at this temperature for ten minutes. The temperature is then increased to 250° C. and stirred at this temperature for 50 min. After cooling to a temperature below 90° C., a brown color liquid mixture is obtained (the color slowly turns deeper after the mixture is exposed to air). The temperature is maintained between 50-70 degrees Celsius, and pressed filtration is carried out to obtain 101 g of brown color solid A. Analysis results show the brown color solid A contains 30% water.

The filtrate is then cooled down to a temperature below 10 degrees Celsius with stirring. Pressed filtration is carried out with the temperature below 10 degrees Celsius. Brown color solid B obtained is 192 gram with 30% of water content.

The brown color solid A is suspended in 200 ml of anhydrous methanol, carbon dioxide is bubble through with vigorous stirring. Pressed filtration is carried out to remove all solids. The filtrate is concentrated to obtain 35.8 g of deep brown color sticky product. The moisture content of this product is less than 1%. The yield of small aromatics obtained is 101% based on lignin weight. Molecular mass analysis shows it is a same mixture as that obtained using calcium oxide, most of them having molecular weights between 162 to 340. Elemental analysis shows the oxygen content of this product is 18%, far below the oxygen content of lignin (˜36%).

The brown color solid B is suspended in 500 ml of anhydrous acetone, carbon dioxide is bubble through with vigorous stirring. Pressed filtration is carried out to remove all solids. The filtrate is concentrated to obtain 73 g of light brown color liquid product. The moisture content of this product is 2.2%. The yield of small organic acids is 102% based on the weight of cellulose and hemicellulose. HPLC analysis of the product shows that only several small organic acids are formed, such as formic acid, glycolic acid, acetic acid, lactic acid, succinic acid, etc. It contains 41 grams of lactic acid, 12 grams of glycolic acid, 14.5 grams of formic acid, 1.3 grams of acetic acid, 3.0 grams of succinic acid, and the rest are 2-hydroxyl isobutyric acid, fumaric acid, etc.

### EXAMPLE 6

To a 1 liter stainless steel autoclave equipped with mechanical stirrer, add 750 ml of distilled water, 112 g of chopped dry grape tree branches (2-10 mm size, containing 38% cellulose, 20% hemicellulose, 29% lignin, and 13% others), 42 g of calcium oxide, and 1 g of disulfonate sodium salt of 9,10-dihydroxyanthracene. The autoclave is sealed, purged with nitrogen for three times, then filled with nitrogen at a pressure of 0.2-1 MPa, heated to 180° C. and maintain at this temperature for ten minutes. The temperature is then increased to 250° C. and stirred at this temperature for 50 min. After cooling to a temperature below 90° C., a brown color liquid mixture is obtained (the color slowly turns deeper after the mixture is exposed to air). The temperature is maintained between 50-70 degrees Celsius, and pressed filtration is carried out to obtain 58 g of brown color solid A and 805 ml of filtrate. Analysis results show the brown color solid A contains 28% water.

The filtrate is then used as solvent for the catalytic depolymerization reaction. 750 ml of filtrate is used, with 112 g of chopped dry grape tree branches, 42 g of calcium oxide, and 1 g of disulfonate sodium salt of 9,10-dihydroxyanthracene. The catalytic depolymerization reaction is carried out using the procedure described in Example 1. 59 g of brown color solid A2 is obtained with 28% water content.

The filtrate is cooled down to the temperature below 10 degrees Celsius with stirring. Pressed filtration is carried out with the temperature below 10 degrees Celsius. Brown color solid B obtained is 248 gram with 30% of water content.

The brown color solid A and A2 are treated separately according to the method described in example 1. The brown color solid A gives 35.4 g of deep brown color sticky product, The brown color solid A2 gives 34.3 g of deep brown color sticky product. The moisture content of these products are less than 1% for both. The yield of small aromatics obtained is the same. Molecular mass analysis shows that they are also same, most of them having molecular weights between 162 to 340. Elemental analysis shows the oxygen content of the products are 18% for both of them. Filtrate can be used as the solvent for the catalytic one-pot hydrolytic depolymerization reaction.

The brown color solid B is suspended in 600 ml of anhydrous acetone, 95% of sulfuric acid is used to adjust pH=1.0 with vigorous stirring. Pressed filtration is carried out to remove all solids. The filtrate is concentrated to obtain 149 g of light brown color liquid product. The moisture content of this product is 2.2%. The yield of small organic acids is 102% based on the weight of cellulose and hemicellulose. HPLC analysis of the product shows that only several small organic acids are formed, such as formic acid, glycolic acid, acetic acid, lactic acid, succinic acid, etc. It contains 78 grams of lactic acid, 25 grams of glycolic acid, 27 grams of formic acid, 4.5 grams of acetic acid, 5.9 grams of succinic acid, and the rest are 2-hydroxyl isobutyric acid, fumaric acid, etc. The result confirmed that filtrate can be used as the solvent for the catalytic depolymerization reaction again.

### EXAMPLE 7

To a 10 liter stainless steel autoclave equipped with mechanical stirrer, add 7500 ml of distilled water, 1120 g of chopped dry grape tree branches (2-10 mm size, containing 38% cellulose, 20% hemicellulose, 29% lignin, and 13% others), 420 g of calcium oxide, and 5 g of disulfonate sodium salt of 9,10-dihydroxyanthracene. The autoclave is sealed, purged with nitrogen three times, then filled with nitrogen at a pressure of 0.2-1 MPa, heated to 180° C. and maintain at this temperature for ten minutes. The temperature is then increased to 250° C. and stirred at this temperature for 50 min. After cooling to the temperature below 90° C., a brown color liquid mixture is obtained (the color slowly turns deeper after the mixture is exposed to air). The temperature is maintained between 50-70 degrees Celsius, and pressed filtration is carried out to obtain 583 g of brown color solid A. Analysis results show the brown color solid A contains 26% water.

The filtrate is then cooled down to a temperature below 10 degrees Celsius with stirring. Pressed filtration is carried out with the temperature below 10 degrees Celsius. Brown color solid B obtained is 1319 grams with 30% of water content.

The brown color solid A is dried under vacuum first, then suspended in 1000 ml of anhydrous methanol, 100 g of dimethyl carbonate is added to the mixture. The mixture is sealed inside 2 liter stainless steel autoclave equipped with mechanical stirrer. The reaction mixture is then been heated to 180 degrees Celsius with vigorous stirring. After two hours reaction, the reaction mixture is cooled to room temperature. Pressed filtration is carried out to remove all solids. The filtrate is distilled to removed methanol and unreacted dimethyl carbonate. A total 380 g of light yellow liquid is obtained via vacuum distillation. The yield is 108% based on lignin weight. The heat of combustion of this light yellow liquid is 43 KJ/g. This value suggests that the light yellow liquid is a good liquid fuel

The brown color solid B is evaluated as deicer compared with conventional deicers. The data is tabulated below.

| | Eutectic | Effective |
|---|---|---|
| Deicer | temperature (° F.) | Temperature (° F.) |
| The brown color solid B | −26 | −12 |
| Sodium chloride | −7 | +15 |
| Calcium chloride | −58 | −25 |
| Potassium acetate | −76 | −15 |

The results show that brown color solid B obtained from the catalytic one-pot hydrolytic depolymerization of lignocellulose is a better deicer than sodium chloride, and is comparable with the expensive potassium acetate. The brown color solid B contains only several small organic acids, such as formic acid, glycolic acid, acetic acid, lactic acid, succinic acid, etc. These small organic acids are all biodegradable.

The results of this example demonstrate that our invented novel process converts lignocelluloses into liquid fuel and fine chemical products using the fewest steps of operation compared with all known processes.

1. A novel process for isolating the products of hydrolysis of lignocellulose in one step operation.

2. The process according to claim 1 in which isolating the products of hydrolysis of lignocellulose in one step operation, comprising of the steps:
(a) providing a lignocellulosic material;
(b) subjecting the lignocellulosic material to a catalytic one-pot, hydrolytic depolymerization in the presence of a alkaline earth metal oxide or hydroxide;
(c) filtrating out the products of hydrolysis of lignocellulose in one step operation; or
(d) filtrating out the products of small molecular aromatics first, and then the products of small organic acids.

3. The process according to claim 1 in which the lignocellulosic material comprises agricultural resides, products produced by plants (herbaceous plant, and ligneous plant), such as leaves, stalks, roots, etc.

4. The process according to claim 2 in which the metal of oxide or hydroxide includes alkaline earth metals and transition metals.

5. The process according to claim 2 in which the ratio of lignocellulose to oxide or hydroxide is in the range of 50 grams of lignocellulose/one molar equivalent of hydroxide anion to 100 grams of lignocellulose/one molar equivalent of hydroxide anion.

6. The process according to claim 2(c) in which the temperature is lower than ten degrees Celsius for the filtration of the products of hydrolysis of lignocellulose in one step operation.

7. The process according to claim 2(d) in which the temperature is higher than fifty degrees Celsius for the filtration of the product of small molecular aromatics first.

8. The process according to claim 2(d) in which the temperature is lower than ten degrees Celsius for the filtration of the products of small organic acids.

9. The process according to claim 2 in which the products of small molecular aromatics are converted into high quality liquid fuels using a one step alkylation or hydrocracking reaction.

10. The process according to claim 2 in which the products of small organic acids can be used as an environmentally benign deicer.

11. The process according to claim 2 in which after the isolation of the product of small molecular aromatics, the filtrate can again be used as the solvent for the catalytic one-pot hydrolytic depolymerization reaction.
